(19) United States
(12) Patent Application Publication
(10) Pub. No.: US 2005/0063948 A1
(43) Pub. Date: Mar. 24, 2005

(54) Methods for targeting interleukin-12 to malignant endothelium

(76) Inventors: Erin B. Dickerson, Madison, WI (US); Stuart C. Helfand, Madison, WI (US); Nasim Akhtar, Madison, WI (US)

Correspondence Address: Licata & Tyrrell P.C., 66 E. Main Street, Marlton, NJ, 08053, US

(21) Appl. No.: 10/917,709

(22) Filed: Aug. 13, 2004

Related U.S. Application Data
(63) Continuation-in-part of application No. 09/801,485, filed on Mar. 8, 2001, now abandoned.

(51) Int. Cl. A61K 38/20; C07H 21/04; C12P 21/04; A61K 39/00; C07K 14/54

(52) U.S. Cl. 424/85.2; 424/192.1; 435/69.52; 435/320.1; 435/325; 530/351; 536/23.5

(57) ABSTRACT

Fusion proteins containing a mammalian interleukin-12 operably linked to an RGD-containing peptide as well as nucleic acid sequences, vectors and host cells for expression of these fusion proteins are provided. Also provided are methods of using these fusion proteins to inhibit tumor growth and to decrease the toxicity associated with interleukin-12 administration.

### INTRODUCTION

This application is a continuation-in-part of U.S. patent application Ser. No. 09/801,485, filed on Mar. 8, 2001 whose contents is incorporated herein by reference in its entirety.

Work presented herein was supported by the National Institutes of Health (Grant No. CA86264) and the U.S. Federal Government may have certain rights in this invention.

### BACKGROUND OF THE INVENTION

The ability of cancer cells to metastasize correlates well with their capacity to initiate angiogenesis, the formation of new blood vessels within tumor tissue (Folkman (1990) J. Natl Cancer Inst. 82:4-6; Folkman (1995) Nature Med. 1:27-31; and Liotta, et al. (1991) Cell 64:327-336). The process of angiogenesis can be inhibited by a number of substances including retinoids, vitamin D, TGF-β, interferons-γ and -α, interleukin-1β, fumagillin and its derivatives AGM-1470 and angiostatin.

Recently interleukin-12 (IL-12) has been reported to have antiangiogenic properties mediated through the induction of IFN-γ and other downstream proteins. IL-12 exhibits a number of activities potentially important in cancer therapy. In humans and mice, IL-12 is a potent activator of natural killer (NK) cell activity (Kobayashi, et al. (1989) J. Exp. Med. 170:827-845) and a major inducer of IFN-γ from NK and T lymphocytes (Chan, et al. (1991) J. Exp. Med. 173:869-879), a cytokine with important immune cell activating capabilities. IFN-γ is also an essential mediator of the antiangiogenic effects ascribed to IL-12 (Voest, et al. (1995) J. Natl Cancer Inst. 87:581-586; Majewski, et al. (1996) J. Invest. Dermatol. 106:1114-1118). IL-12 enhances tumor cell killing mediated by immune cells specifically directed toward tumor targets by antitumor antibodies (antibody-dependent cellular cytotoxicity, ADCC) (Lieberman, et al. (1991) J. Surg. Res. 50:410-415). IL-12 stimulates nitric oxide production in vivo, resulting in delayed tumor progression in mice (Wigginton, et al. (1996) Cancer Res. 56:1131-1136). Endogenous IL-12 production has also been documented to gradually diminish as tumor burden increases (Handel-Fernandez, et al. (1997) J. Immunol. 158:280-286), thus forming a rationale for providing IL-12 to cancer patients to reconstitute cell-mediated antitumor responses. IL-12 is also a potent inhibitor of tumor-driven angiogenesis (Voest, et al. (1995) supra; Majewski, et al. (1996) supra) demonstrating significant in vivo inhibition of tumor blood vessel formation in mice mediated through IFN-γ inducible protein-10 (IP-10; Sgadari, et al. (1996) Blood 87:3877-3882), a chemokine that has a potent antiangiogenic effect on the vasculature of growing tumors (Angiolillo, et al. (1996) Ann NY Acad. Sci. 795:158-167; Arenberg, et al. (1996) J. Exp. Med. 184:981-992). In vitro, it inhibits the formation of tube-like structures by endothelial cells (Angiolillo, et al. (1995) J. Exp. Med. 182:155-162). In vivo, induction of IP-10 by IL-12 results in central tumor necrosis with surrounding blood vessels showing intimal thickening, endothelial cell apoptosis, and partial to complete occlusion of the vessel lumens by thrombosis (Angiolillo, et al. (1996) supra; Dias, et al. (1998) Int. J. Cancer 75:151-157). IP-10 is a chemoattractant for T cells and monocytes, supporting a role for it in leukocyte recruitment (Luster and Leder (1993) J. Exp. Med. 178:1057-1065; Taub, et al. (1993) J. Exp. Med. 177:1809-1814). More recently, IL-12 has been shown to exert antiangiogenic effects through its role as a regulator of VEGF and matrix metalloproteinase (MMP) production (Dias, et al. (1998) Int. J. Cancer 78:361-365). In addition, IL-12 has been shown to synergize with IL-2 to exert an antiangiogenic effect (Watanabe, et al. (1997) Am. J. Pathol. 150:1869-1880), thereby depriving growing tumors of essential blood supply (Auerbach and Auerbach (1994) Pharmac. Ther. 63:265-311; Gasparini (1997) Crit. Rev. One. Hematol. 26:147-162).

In preclinical studies, recombinant IL-12 was shown to mediate destruction of established tumors in mice (Dias, et al. (1998) supra; Brunda, et al. (1993) J. Exp. Med. 178:1223-1230; Nastala, et al. (1994) J. Immunol. 153:1697-1706; Zou, et al. (1995) Intl. Immunol. 7:1135-1145; O'Toole, et al. (1993) J. Immunol. 150:294) and also to exert an antimetastatic effect, especially when combined with IL-2 (Wigginton, et al. (1996) Cancer Res. 56:1131-1136).

However, like many cancer therapies, a major obstacle to IL-12 therapy has been its appreciable toxicity, including death, in humans (Soiffer, et al. (1993) Blood 82:2790-2796; Atkins, et al. (1997) Clin. Cancer Res. 3:409-417; Leonard, et al. (1997) Blood 90:2541-2548; Robertson, et al. (1999) Clin. Cancer Res. 5:9-16). Attempts have been made to alter the dosing regime to downregulate the extreme and life-threatening systemic IFN-γ peak that follows multi-day repetitive IL-12 dosing in humans (Atkins, et al. (1997) supra; Leonard, et al. (1997) supra). While better tolerated clinically, however, this altered dosing regime results in inferior tumor control in mice (Coughlin, et al. (1997) Cancer Res. 57:2460-2467).

Various approaches for reducing toxicity by targeting anticancer agents to the tumor tissue have been described. For example, a chemotherapeutic drug is linked to a ligand specific for a binding partner expressed only on the surface of tumor cells. Such ligands have included monoclonal antibodies and peptides.

For example, the expression of αᵥβ₃ in tumor-bearing animals has been shown to be a specific marker for tumor neovasculature and the dependence of angiogenic endothelium on αᵥβ₃ comprising tumor vasculature have combined to make αᵥβ₃ an important marker for cancer therapy. Targeting of αᵥβ₃ on tumor vasculature has been accomplished using antagonists such as monoclonal antibody LM609 (Brooks, et al. (1994) Cell 79:1157-1164) or peptides with αᵥβ₃ binding specificity such as RGD (Arap, et al. (1998) Science 279:377-380). In addition, a combination of an antitumor antibody-IL-2 fusion protein plus an αᵥβ₃ antagonist has proven better than either monotherapy in controlling murine syngeneic melanoma, colon carcinoma and neuroblastoma (Lode, et al. (1999) Proc. Natl. Acad. Sci. USA 96:1591-1596).

Further, short peptides containing the RGD sequence have been shown to inhibit in vitro tumor cell invasion and in vivo tumor dissemination (Ruoslahti (1992) Br. J. Cancer 66:239-242). One RGD peptide containing 4 cysteines (i.e., Ala-Cys-Asp-Cys-Arg-Gly-Asp-Cys-Phe-Cys-Gly (SEQ ID NO:1) was shown to be particularly potent at inhibiting αᵥβ₃-mediated cell attachment to vitronectin (Koivunen, et al. (1995) Biotechnology 13:265-270). A truncated form of this peptide, specifically, Cys-Asp-Cys-Arg-Gly-Asp-Cys-Phe-Cys (SEQ ID NO:2) has been shown to be specific in homing to vasculature of various tumors including carcinoma, sarcoma and melanoma and doxorubicin linked to this RGD proved to be highly effective in limiting tumor growth in vivo (Arap (1998) Science 279:377-380; Koivunen, et al. (1995) supra).

WO 2000/47228 discloses a chemotherapeutic comprising an angiogenesis inhibiting agent, preferably an αᵥβ₃ antagonist such as an RGD-containing peptide, an antibody having antigen binding specificity for αᵥβ₃ or the αᵥβ₃ receptor, or an αᵥβ₃ mimetic, and an anti-tumor immunotherapeutic agent with a cell-effector component, preferably IL-2, and a tumor-associated antigen targeting component.

Novel approaches to deliver IL-12 safely while retaining its immunostimulatory, antiangiogenic, and antitumor properties are highly desirable. The present invention meets this long-felt need in providing RGD-containing peptides to target IL-12 to angiogenic endothelial cells and αᵥβ₃-Positive tumor cells.

### SUMMARY OF THE INVENTION

The present invention relates to a fusion protein containing interleukin-12 operably linked to an RGD-containing peptide and methods for using the same.

One embodiment of the present invention is a method for inhibiting growth of an angiogenic endothelial cell or an αᵥβ₃-positive tumor cell. The method involves delivering a mammalian interleukin-12 protein to an angiogenic endothelial cell or an αᵥβ₃-positive tumor cell via a fusion protein containing interleukin-12 operably linked to an RGD-containing peptide thereby inhibiting the growth of the angiogenic endothelial cell or the αᵥβ₃-positive tumor.

Another embodiment of the present invention is a method for decreasing toxic side effects associated with interleukin-12 administration in a mammal. This method of the invention involves generating a fusion protein comprising interleukin-12 and an RGD-containing peptide, and administering to a mammal the fusion protein thereby decreasing the toxic side effects associated with interleukin-12.

A further embodiment of the present invention is a method of treating cancer in a mammal. This method of the invention involves administering to a mammal having a cancer an effective amount of a fusion protein composed of interleukin-12 operably linked to an RGD-containing peptide so that the cancer in the mammal is treated.

### DETAILED DESCRIPTION OF THE INVENTION

The present invention relates to a bifunctional fusion protein which targets αᵥβ₃ expressed on the neovasculature of tumors and αᵥβ₃-positive tumor cells. The bifunctional fusion protein of the present invention contains the antiangiogenic cytokine interleukin-12 (IL-12) or a biologically active fragment or variant thereof operably linked to a vascular homing peptide such as a small peptide arginine-glycine-aspartate (RGD) with the ability to inhibit proliferation of cancer cells expressing αᵥβ₃, and to inhibit growth and development of angiogenic αᵥβ₃-expresssing endothelial cells serving the tumors.

For purposes of the present invention, by “vascular homing peptide”, it is meant, a peptide comprising the amino acid sequence arginine-glycine-aspartic acid (RGD), also referred to herein as a “RGD-containing peptide”. In simplest form, this peptide may contain only the amino acids RGD. However, this peptide may further include additional amino acids which do not interfere with, and desirably enhance, the ability of the peptide to target αᵥβ₃. In particular embodiments, the vascular homing peptide encompasses the amino acid sequence Cys-Asp-Cys-Arg-Gly-Asp-Cys-Phe-Cys (SEQ ID NO:2), also referred to herein as RGD-4C.

By “biologically active fragment or variant thereof”, it is meant fragments or variants of a full-length mammalian IL-12 protein which exhibit the same or similar antiangiogenic or antitumor activity of the full-length IL-12 protein. By “variant” it is meant a polypeptide which differs in amino acid sequence from IL-12 by one or more substitutions, additions, deletions, fusions or truncations, or any combination thereof, but which exhibits similar activity to IL-12. Substitutions which do not significantly affect the activity of a protein are well-known to the skilled artisan and can, for example, be based on Dayhoff's mutation odds matrix (Bordo and Argos (1991) J. Mol. Biol. 217:721-729), the PAM250 scoring matrix (Pearson (1990) Meth. Enzymol. 183:63-98), or based on the location of the amino acids, i.e., exposed to solvent (Taylor (1986) J. Theor. Biol. 119:205-218) or interior to the protein (Bordo and Argos (1991) supra).

By “operably linked” it is meant that the IL-12 is attached to the vascular homing peptide as one contiguous amino acid sequence in a manner which maintains both the biological activity of IL-12 and the targeting ability of the vascular homing peptide for αᵥβ₃. For example, in one embodiment, RGD-4C is linked to the carboxy-terminus of the p35 or p40 subunit of the antiangiogenic cytokine IL-12. Alternatively, the RGD-containing peptide can be linked to an N-terminus of either subunit of a mammalian IL-12. Further, as will be understood by one of skill in the art upon reading this disclosure, the RGD-containing peptide sequence and IL-12 can be linked at other positions in each of the sequences so long as the biological activity of IL-12 and the targeting ability of the RGD-containing peptide for αᵥβ₃ are maintained. For example, since IL-12 is a heterodimer, the RGD-containing peptide can also be placed on the C-terminal ends of the leader sequences which are present on the N-termini of the p35 and p40 subunits. Maintenance of these activities and abilities can be ascertained routinely by those of skill in the art in accordance with the methods described herein.

The present invention also relates to nucleic acid sequences encoding bifunctional fusion proteins containing mammalian IL-12 or a biologically active fragment or variant thereof operably linked to a vascular homing peptide, as well as vectors and host cells containing these nucleic acid sequences which encode the bifunctional fusion proteins. Nucleic acid sequences for IL-12 from various mammals including, but not limited to, mouse, rat, woodchuck, dog, goat, sheep, red deer and human, are described in GENBANK. Such sequences can be ligated to a nucleic acid sequence encoding an RGD-containing peptide routinely in accordance with well-known methods. Alternatively, for shorter peptides such as those containing only RGD, incorporation can by done by polymerase chain reaction (PCR).

By way of illustration, RGD-4C was fused to the C-terminus of the p35 subunit of murine IL-12 to generate p35RGD-4C. Nucleic acid sequences encoding the p35RGD-4C fusion protein were cloned into a mammalian expression vector and co-expressed with p40 nucleic acid sequences in CHO cells to produce an IL-12 protein containing the RGD vascular homing peptide, referred to herein as mrIL-12vp. Secretion of mrIL-12vp into culture supernatants by transfected CHO cell clones was determined by an ELISA specific for complete IL-12 (p70) protein. Eight clones producing high concentrations of mrIL-12vp were expanded, and the production of fusion protein over 24 hours was determined. Two clones, C2-A4 and C2-B3, produced approximately 1400 ng and 900 ng/million cells/24 hours, respectively. The concentration of mrIL-12vp in the cell culture supernatants ranged from 0.8-1.2 μg/mL. Clone C2-A4 was used for further production of the fusion protein, and mrIL-12vp from cell culture supernatants was purified by affinity chromatography. The purity of the mrIL-12vp was confirmed by SDS-PAGE. In addition, immunoblot analysis was performed to verify the presence of both the p35 and p40 subunits of IL-12. Fractions obtained from non-transfected CHO cell culture supernatants subjected to an identical purification scheme did not have any detectable IL-12 when assessed by an ELISA specific for the p70 form of IL-12 To verify that mrIL-12vp maintained biological activity consistent with that of mrIL-12, induction of IFN-γ by immune cells, a surrogate marker of IL-12 activity, was determined by stimulating activated mouse splenocytes with mrIL-12 or mrIL-12vp (0.01-10 ng/mL) for 48 hours. An ELISA was used to quantitate IFN-γ in the culture supernatants, and these values were used to determine the ED₅₀ of mrIL-12vp. Reported ED₅₀ values for commercial preparations of mrIL-12 from three different sources ranged from 10 to 200 μg/mL, and these values were confirmed herein. For the mrIL-12 used throughout the studies disclosed herein, the ED₅₀ was 100-150 μg/mL. The ED₅₀ for purified mrIL-12vp was moderately higher, approximately 800 μg/mL. The observed decrease in biological activity may be due to the modification at the C-terminal end of the p35 subunit by the addition of the RGD-4C peptide. A similar decrease in IL-12 biological activity was observed when the C-terminus of the p35 subunit was linked to the N-terminus of the p40 subunit by a short amino acid linker (Lieschke, et al. (1997) Nat. Biotechnol. 15:35-40). Representative fractions from non-transfected CHO cell culture supernatants purified in a manner identical to those fractions of mrIL-12vp did not show IFN-γ production.

To determine the activity of mrIL-12vp in vivo, the IFN-γ concentrations in sera from BALB/c mice treated with mrIL-12 or mrIL-12vp (1 μg/mouse/day) by continuous subcutaneous (SC) infusion were compared with serum levels in mice treated with phosphate-buffered saline (PBS). IFN-γ peaked three days after initiating treatment with either mrIL-12 or mrIL-12vp reaching levels>600 μg/mL compared to control mice treated with PBS (<100 μg/mL). The concentration of IFN-γ in the sera of mice treated with mrIL-12 did not differ significantly from the levels found in mice treated with mrIL-12vp (P>0.28). These results indicate that mrIL-12vp maintains biological activity in vivo comparable to that of mrIL-12.

The specificity of mrIL-12vp binding to αᵥβ₃ integrin-positive cell lines was subsequently determined. M21, a human melanoma line which expresses αᵥβ₃ integrin on its surface (Felding-Habermann, et al. (1992) J. Clin. Invest. 89:2018-2022), was used as a αᵥβ₃ integrin-positive cell line for this analysis. Prior to the studies with mrIL-12vp, the expression of αᵥβ₃ integrin was confirmed based on intense labeling of M21 cells with the anti-αᵥβ₃ integrin antibody LM609 using flow cytometry. In contrast, Saos-2, a human osteosarcoma line, showed almost no expression of the integrin. Immunofluorescence evaluation of cells grown on chamber slides confirmed specific binding of mrIL-12vp, but not mrIL-12 to M21 cells. There was no binding of mrIL-12 or mrIL-12vp to Saos-2 cells. When the primary antibody recognizing IL-12 p40 was omitted from the staining sequence, the labeling intensity was comparable to controls (i.e., Saos-2).

One of the major obstacles to cytokine therapy with IL-12 is its appreciable toxicity when administered systemically (Leonard, et al. (1997) supra; Soiffer, et al. (1993) supra; Atkins, et al. (1997) supra; Robertson, et al. (1999) Clin. Cancer Res. 5:9-16). In mice, IL-12 dosing protocols were developed to avoid pulmonary edema observed when mice were treated with repetitive daily doses of IL-12 without interruption (Trinchieri (1998) Adv. Immunol. 70:83-243). Using these schedules, many mouse strains are able to tolerate repeated injections of 1 μg of IL-12/day, but some strains succumb to this dose and can only withstand doses 5-10 times lower (Coughlin, et al. (1997) supra). To assess the toxicity of mrIL-12vp, strain DBA/2J mice, a mouse stain known to be more sensitive to IL-12 toxicity, was used so that differences between mrIL-12 and mrIL-12vp would be readily observed. Intraperitoneal (IP) injections of mrIL-12 ranged from 0.025-0.5 μg/mouse/day. The maximum tolerated dose (MTD) for mrIL-12 by IP injection was determined to be 0.025 μg/mouse/day. Higher doses caused severe side effects including sudden death after seven days. Signs of toxicity including inappetence, ruffled fur, listlessness, weight loss, labored breathing, and pulmonary edema were readily apparent. Signs of toxicity were not observed in mice treated with comparable doses of mrIL-12vp (IP), and histological examination revealed little or no pulmonary edema. In addition, there were no sudden deaths in this group.

Further, mrIL-12 and mrIL-12vp were administered by continuous SC infusion via surgically implanted osmotic pumps. By changing the route and schedule of administration, the MTD delivered by the osmotic pumps was 0.5 μg/mouse/day of mrIL-12 or mrIL-12vp, 20 times more than the MTD (0.025 μg/mouse/day) observed for mrIL-12 by IP administration. There were no observable side effects in the mice given mrIL-12vp. However, histological evaluation of the livers from DBA/2J mice given 0.5 μg/mouse of IL-12/day by continuous infusion showed focal necrotizing hepatitis while the mrIL-12vp-treated mice did not have any liver lesions. The dose of mrIL-12 or mrIL-12vp was not increased beyond the 0.5 μg/mouse/day dose since evidence of toxicity was confirmed for mrIL-12. Thus, in both models, mrIL-12vp was less toxic than mrIL-12 and the route and schedule of delivery of IL-12 may be of importance.

To determine whether mrIL-12vp enhances the antiangiogenic effect of IL-12, sponges containing bFGF (100 ng) were surgically implanted into an avascular area of the right cornea of adult BALB/c mice to induce vessel growth. Two days after sponge implantation, osmotic pumps were placed SC in the mice to deliver a seven-day treatment of mrIL-12, mrIL-12vp, or PBS by continuous infusion. The results of this experiment are presented in Table 1.

TABLE 1

| | | % Corneal | |
|---|---|---|---|
| | Concentration | Surface Area | |
| | (IL-12 | Occupied by | % Decrease |
| Treatment | equivalent) | Vessels (Mean) | vs. Control |
| Control | — | 43.4 ± 2.1 | — |
| MrIL-12 | 0.25 μg | 36.7 ± 6.2 | 15.5% |
| MrIL-12vp | 0.25 μg | 26.7 ± 4.7 | 38.5%^{ad} |
| Control | — | 33.8 ± 11.0 | — |
| MrIL-12 | 0.5 μg | 29.3 ± 6.2 | 13.3% |
| MrIL-12vp | 0.5 μg | 11.2 ± 3.0 | 66.9%^{ab} |
| Control | — | 36.7 ± 18.2 | — |
| Sham^{c} | — | 33.5 ± 10.0 | 8.7% |
| MrIL-12 | 1 μg | 16.5 ± 1.1 | 55.0%^{a} |
| MrIL-12vp | 1 μg | 6.6 ± 1.0 | 82.0%^{ab} |

^{a}P < 0.05 compared with control treated mice.
^{b}P < 0.05 compared with mrIL-12-treated mice.
^{c}Mice treated with representative cell culture supernatant fractions from non-transfected CHO cells.

In the PBS-treated mice, large vessels were observed growing towards the sponge, and smaller, densely packed vessels were growing around the sponge. The corneal vascular density of mice treated with the two lower doses (0.25 and 0.5 μg/mouse/day) of IL-12 did not differ from controls (P>0.05) (Table 1). Mice treated with the highest dose of mrIL-12 (1 μg/mouse/day), had noticeable inhibition of vessel growth, and there was a significant decrease (55%, P=0.05) in the vessel area covering the corneas from mice in this treatment group. In contrast, mice treated with mrIL-12vp showed a marked decrease in corneal vessel surface area at all doses tested (0.25, 0.5, and 1.0 μg/mouse/day). The lowest dose of 0.25 μg/mouse/day showed a 39% decrease in the vessel surface area when treated mice were compared to controls (P=0.04). At the highest dose of mrIL-12vp, suppression of corneal angiogenesis was almost complete, with an 82% decrease in the vessel surface area (P=0.05). These results indicate that mrIL-12vp significantly enhances the antiangiogenic effect of mrIL-12. Similar results were obtained in identical experiments using VEGF (200 ng) as the inducer of neovascularization and treatment with mrIL-12 or mrIL-12vp at a dose of 1 μg/mouse/day.

Corneal neovascularization assays were also carried out in DBA/2J mice treated with 0.5 μg/mouse/day of mrIL-12, mrIL-12vp, or with PBS by subcutaneous continuous infusion. Similar to BALB/c mice treated with 0.5 μg/day of mrIL-12, the surface area of DBA/2J mice treated with this dose of mrIL-12 was comparable to that of control mice. In contrast, the corneas of DBA/2J mice treated with 0.5 μg/mouse day of mrIL-12vp showed markedly reduced neovascularization confined to the limbal region. Thus, the antiangiogenic effect of mrIL-12vp in DBA/2J mice was even more striking than the response observed in BALB/c mice. Representative fractions from nontransfected CHO cells did not have angiosuppressive effects (Sham, Table 1).

The capacity of the RGD-4C peptide alone to inhibit angiogenesis, and its contribution to the enhanced antiangiogenic effect observed with mrIL-12vp was determined. In corneal angiogenesis assays, RGD-4C was administered to mice for seven days at a molar concentration equivalent to that present in 1 μg of mrIL-12vp, and an antiangiogenic effect was not observed. The lack of antiangiogenic activity observed in the corneal neovascular assay in mice was most likely attributable to the use of a relatively low dose of RGD-4C. Thus, a direct comparison between free RGD peptide and RGD peptide bound to IL-12 is not directly comparable and a means to determine the concentration of RGD-4C was found.

To further elucidate the antiangiogenic contribution of the RGD-4C peptide in the fusion protein, the biological activity of IL-12 was eliminated while maintaining the activity of RGD-4C. To accomplish this, two strategies were used. First IFN-γ^{-/-} mice were used in corneal neovascular assays. IFN-γ is a critical and potent requisite downstream mediator of IL-12-triggered antiangiogenesis pathways (Voest, et al (1995) supra; Majewski, et al (1996) supra). IFN-γ^{-/-} mice were treated with 1 μg/mouse/day of mrIL-12, mrIL-12vp or PBS by continuous infusion. Even though IFN-γ was completely absent in these mice, as determined by ELISA, inhibition of angiogenesis in mice treated with mrIL-12 was 27%, while inhibition of angiogenesis observed in mrIL-12vp mice was 45%. In multiple experiments, an antiangiogenic effect was consistently observed in both mrIL-12 and mrIL-12vp treated IFN-γ^{-/-} mice indicating that this response may not depend entirely upon the presence of IFN-γ. In addition, the antiangiogenic effects of mrIL-12vp were superior to those of mrIL-12 in each experiment, indicating a role for the RGD-4C peptide in mrIL-12vp in mediating these effects.

The second strategy was to eliminate signaling of IL-12 through it receptors and nullify the biological activity of IL-12 allowing the biological activity of RGD-4C to be examined independently. To achieve this, IL-12R^{-/-} mice were used in corneal neovascular assays. Mice were treated with 1 μg/mouse/day of mrIL-12 or mrIL-12vp for seven days by continuous infusion. Mice treated with mrIL-12vp showed a significant decrease in neovasculature (P<0.05) when compared with the antiangiogenic activity in mrIL-12-treated mice. The overall decrease in multiple experiments was 25-30%. Corneas in mice treated with mrIL-12 did not differ significantly from controls. This result indicates RGD-4C has antiangiogenic activity independent from that of IL-12, and the use of this model for separating the IL-12 biological activity from that of RGD-4C addresses the unique role for the RGD-4C moiety in mrIL-12vp. These results further indicate that RGD-4C fused to IL-12 contributes to the antiangiogenic effects of mrIL-12vp.

Because of the significant antiangiogenic effect observed in the corneal neovascular assay in BALB/c mice treated with mrIL-12vp, it was determined whether mrIL-12vp could inhibit tumor growth in a murine tumor model. The NXS2 tumor model retains many features of human neuroblastoma including high GD2 and tyrosine hydroxylase expression, and metastatic growth to liver and bone marrow (Lode, et al. (1997) J. Natl. Cancer Inst. 89:1586-1594). The injection of 2×10⁶ tumor cells into the lateral flank results in a palpable tumor within 9 to 11 days. Experimental metastases can be induced by injection of cells into the tail vein or by resection of the primary tumor approximately 18 days after implantation.

Before beginning the tumor studies, NXS2 cells were analyzed in the corneal neovascular assay to determine if the cells produced an angiogenic response, and if this angiogenic response could be inhibited by continuous infusion of either mrIL-12 or mrIL-12vp. NXS2 cells placed on a polyvinyl sponge and put into a corneal pocket caused a robust growth of neovessels sprouting from the limbal region and reaching the sponge within eight days. To ascertain the antiangiogenic effects of either mrIL-12 or mrIL-12vp on NXS2 cells, eight-week old female A/J mice receiving NXS2 corneal implants were treated with either PBS or 0.5 μg/mouse/day of mrIL-12 or mrIL-12vp starting two days after placement of the sponges. A/J mice treated for seven days with mrIL-12vp showed a significant decrease in neovasculature (P=0.02) when compared with the antiangiogenic activity in mrIL-12-treated mice or with controls. Angiogenic inhibition in mrIL-12vp-treated mice was complete while inhibition in mrIL-12 was 80%. The antiangiogenic response generated by both mrIL-12 and mrIL-12vp was greater in A/J mice when compared to both BALB/c and DBA/2J mice at the 0.5 μg/mouse/day dose. This difference may reflect the sensitivity of certain strains of mice to IL-12.

To determine if mrIL-12vp could inhibit or slow the growth of NXS2 tumors in A/J mice, mice were injected with 2×10⁶ NXS2 cells in the right lateral flank. When tumors were palpable (9-11 days later), mice were treated with 1 μg/mouse/day of mrIL-12 or mrIL-12vp by continuous infusion for 3 weeks. For these studies, pumps capable of delivering material for 28 days were used instead of pumps having a 7 day capacity. Tumors were measured on a weekly basis. Mice treated with mrIL-12vp showed a significant slowing of tumor growth (P=0.03) when compared with controls or mrIL-12-treated mice (Table 2).

TABLE 2

| | Tumor Volume (mm³) | | |
|---|---|---|---|
| Week | PBS | mrIL-12 | mrIL-12 |
| 1 | 197 ± 55 | 246 ± 46 | 109 ± 58 |
| 2 | 995 ± 295 | 909 ± 243 | 298 ± 86 |
| 3 | 1648 ± 176 | 1374 ± 187 | 637 ± 261 |

While the fusion proteins disclosed herein contain RGD sequences of nine amino acids in length, fusion proteins containing a shorter (e.g., containing only Arg-Gly-Asp) or longer (e.g., Ala-Cys-Asp-Cys-Arg-Gly-Asp-Cys-Phe-Cys-Gly; SEQ ID NO:1) peptide are also contemplated. For these fusion proteins, the cloning and expression of the p40 subunit is according to that described herein. To incorporate RGD into, for example, the C-terminal end of a p35 subunit, the nucleotide sequence for RGD can be directly incorporated into an antisense primer of p35 provided herein. Desirably, these primers also contain the proper restriction enzyme sites. The final product can be amplified by 35 rounds of PCR and ligated, cloned, selected, and expressed as disclosed herein.

Further, as will be understood by those of skill in the art upon reading this disclosure, other methods than exemplified herein for production of plasmids containing nucleic acid sequences for a mammalian IL-12 or biologically active fragments or variants thereof and methods for ligating a nucleic acid sequence encoding an RGD-containing peptide to the plasmid containing the mammalian IL-12 nucleic acid sequence, as well as other vectors and host cells for expression of the bifunctional fusion proteins, can be used and are well-established in the art and/or are commercially available.

The presence of the IL-12 nucleic acids in the plasmid and DNA encoding the RGD-containing peptide following ligation to a plasmid comprising a mammalian IL-12 nucleic acid sequence can be verified by routine sequencing. Desirably, host cells for expression of the fusion protein are mammalian cells expressing only low levels or no αᵥβ₃. Examples include, but are not limited to, CHO cells and Saos-2 cells. Other cells expressing low levels of or no αᵥβ₃ can be routinely identified by immunoassays and the like.

The results provided herein are the first demonstration that fusion proteins containing a mammalian IL-12 operably linked to a RGD-containing peptide provide a useful means for targeting IL-12 to tumor cells. As also demonstrated herein, the fusion proteins of the present invention inhibit angiogenesis as well as tumor cell growth in established in vitro and in vivo models. Accordingly, the fusion proteins of the present invention provide a useful means for treating tumor vasculature and tumors expressing αᵥβ₃ via IL-12 and for lowering the toxicity associated with IL-12 in mammals. By linking RGD-4C to IL-12, the half-life of RGD-4C can be extended dramatically prolonging the duration of its activity and reducing the toxicity of IL-12, therefore making this fusion protein useful in the treatment of cancer.

Therefore, the present invention is a method for treating a cancer in a mammal, in particular a mammal with tumors cells expressing αᵥβ₃ (e.g., melanoma, breast cancer, prostate cancer, and the like) using a mammalian IL-12 operably linked to an RGD-containing peptide. A mammal having a cancer may exhibit one or more of the typical signs or symptoms associated with the disease including a lump, high PSA levels, feelings of weakness, and increased pain perception. To treat the cancer, the mammal is administered an effective amount of a mammalian IL-12 operably linked to an RGD-containing peptide to have a beneficial or desired clinical result. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. Treatment can also mean prolonging survival as compared to expected survival if not receiving treatment. As will be understood by the skilled artisan, the signs or symptoms of the cancer can vary with the stage of the cancer and the signs or symptoms associated with various stages are well-known to the skilled clinician. See, for example, The American Joint Committee on Cancer Staging Manual, Sixth Edition.

For purposes of the present invention, by “mammal” it is meant to be inclusive, but not limited to, humans and veterinary animals (e.g., domestic pets, sport animals, laboratory animals, and livestock). Dosing regimes for the fusion proteins of the present invention can be routinely determined in accordance with pharmacological activity data from experiments in in vitro and in vivo models such as described herein as well as previously established dosing regimes for IL-12.

It is contemplated that the mammalian IL-12 operably linked to an RGD-containing peptide is formulated into a pharmaceutical composition comprising an effective amount of the fusion protein and a pharmaceutically acceptable carrier. Pharmaceutically acceptable carriers are materials useful for the purpose of administering the medicament, which are preferably sterile and non-toxic, and can be solid, liquid, or gaseous materials, which are otherwise inert and medically acceptable, and are compatible with the active ingredients.

The pharmaceutical compositions can contain other active ingredients such as preservatives and can take the form of a solution, emulsion, suspension, ointment, cream, granule, powder, drops, spray, tablet, capsule, sachet, lozenge, ampoule, pessary, or suppository. The pharmaceutical compositions be administered by continuous or intermittent infusion, parenterally, intramuscularly, subcutaneously, intravenously, intra-arterially, intrathecally, intraarticularly, transdermally, orally, bucally, as a suppository or pessary, topically, as an aerosol, spray, or drops, depending upon whether the preparation is used to treat internal or external cancers. Such administration can be accompanied by pharmacologic studies to determine the optimal dose and schedule and would be within the skill of the ordinary practitioners (e.g., amounts to be administered can be primarily based on the IL-12 equivalent of the fusion protein). In particular embodiments, the fusion protein is diluted and delivered in a saline solution via a subcutaneous pump. In alternative embodiment, intravenous administration is performed.

Having shown that the fusion protein of the present invention inhibits growth of angiogenic endothelial cells and αᵥβ₃-positive cells, the present invention also relates to a method for using a fusion protein of the invention to inhibit the growth of angiogenic endothelial cells or αᵥβ₃-positive cells either in vitro or in vivo. The method involves delivering a mammalian interleukin-12 protein to an angiogenic endothelial cell or an αᵥβ₃-positive tumor cell via a fusion protein of the present invention thereby inhibiting the growth of the angiogenic endothelial cell or the αᵥβ₃-positive tumor. A reduction or inhibition of cell growth is intended to mean a 30%, 40%, 50%, 60%, 70%, 80%, 90%, 95%, 98%, or 100% decrease when compared to otherwise same conditions wherein the fusion protein is not present. As one of skill in the art can appreciate, means for determining cell growth can vary depending on whether the cell is in vitro or in vivo. For example, cell growth measurements in vitro can be determined by counting cells before and after the addition of the fusion protein and comparing the number of cells present after addition of fusion protein to similar cells not receiving the fusion protein. Similarly, cell growth measurements in vivo can be determined by monitoring the size of a tumor before and after delivery of the fusion protein.

The following non-limiting examples are provided to further illustrate the present invention.

### EXAMPLE 1

### Materials

Cytokines. Murine recombinant IL-12, basic fibroblast growth factor (bFGF) and vascular endothelial cell growth factor (VEGF) were purchased from Peprotech (Rocky Hill, N.J.). Murine recombinant IL-12 was purchased from Biosource (Camarillo, Calif.) and R&D Systems (Minneapolis, Minn.).

Mice. BALB/c, DBA/2J, IFN-γ^{-/-}, and IL-12R^{-/-}mice were obtained from Jackson Laboratory (Bar Harbor, Me.). A/J mice were obtained from Harlan Sprague Dawley (Indianapolis, Ind.). Animals were bred and housed according to well-established methods.

Tumor Cells. M21 human melanoma cells are well-established in the art (Felding-Habermann, et al. (1992) supra), and Saos-2 osteosarcoma cells were purchased from ATCC (Manassas, Va.). NXS2 murine neuroblastoma cells are also well-known to the skilled artisan (Lode, et al. (1997) supra).

Antibodies. LM609 (Chemicon International, Temecula, Calif.) is a murine monoclonal IgG₁ isotype that recognizes the αᵥβ₃ integrin heterodimer. Antimouse IL-12 p40 was purchased from Santa Cruz Biotechnology (Santa Cruz, Calif.), and a goat antirabbit IgG linked to fluorescein isothiocyanate (FITC), was purchased from Sigma (St. Louis, Mo.) and used in the immunofluorescence labeling experiments. Goat antimouse IgG conjugated to FITC (BD PHARMINGEN™) was used for the flow cytometry experiments.

Flow cytometry employing antibody LM609 was used to assess the expression of αᵥβ₃ integrin on the surface of M21 and Saos-2 cells according to standard methods (Helfand, et al. Cancer Res. 59: 3119-3127). Irrelevant murine IgG₁ was used as an isotype control antibody.

### EXAMPLE 2

### Cloning and Expression of mrIL-12vp

The cDNA encoding the p40 subunit of murine IL-12 was amplified by PCR using a vector containing the cDNA for the murine p40 subunit. The primer sets (Operon, Alameda, Calif.) for the p40 cDNA were 5′-CCG GTA CCA TGT GTC TTC AGA AGC TA-3′ (sense; SEQ ID NO:3) and 5′-CCG ATA TCC TAG GAT CGG ACC CTG CA-3′ (antisense; SEQ ID NO:4). Nucleic acid sequences encoding the p40 subunit were amplified by 35 rounds of PCR with an annealing temperature of 60 C using standard PCR reagents and conditions. The PCR product was ligated into the vector pcDNA3.1/myc-HisA (INVITROGEN™ Life Technologies, Carlsbad, Calif.) using the KpnI and EcoRV restriction sites which were also incorporated into the sense and antisense primers, respectively. E. coli strain JM109 was then transformed with the vector and the resulting colonies checked for the presence of the plasmid containing the p40 cDNA insert. Chinese hamster ovary (CHO) cells were transfected with endotoxin-free plasmid (ENDOFREE® Plasmid Maxi Kit, QIAGEN®, Valencia, Calif.) using SUPERFECT® (QIAGEN®). Positive clones were selected with G418 (300 μg/mL) and individual colonies arising from single cells were isolated by limiting dilution and expanded. Supernatants were tested for the murine IL-12 p40 subunit using an OPTEIA™ ELISA kit (BD PHARMINGEN™, San Diego, Calif.). Clones producing the highest p40 concentrations (C2 and C4) were used for transfection with a second vector containing the cDNA encoding the murine p35 subunit.

For amplification of the p35 subunit cDNA, RNA was extracted from the spleens of 8-12 week old female BALB/c mice using TRIZOL® (INVITROGEN™) in accordance with the manufacturer's specifications. The RNA was purified using RNEASY® spin columns (QIAGEN®) The RNA was reverse-transcribed using a SUPERSCRIPT™ II RT-PCR kit (INVITROGEN™) in accordance with the manufacturer's instructions. Nucleic acid sequences encoding the p35 subunit were amplified by 35 rounds of PCR using the primers 5′-CCG GTA CCA TGT GTC AAT CAC GTC TAC-3′ (sense; SEQ ID NO:5) and 5′-CCG ATA TCT CAG GCG GAG CTC AGA TA-3′ (antisense; SEQ ID NO:6) under standard conditions. The product was ligated into the vector pSP72 (PROMEGA®, Madison, Wis.) using the restriction sites KpnI and EcoRV, which were also incorporated into the sense and antisense primers, respectively. E. coli strain JM109 was transfected with the vector, the plasmid was purified from colonies selected on ampicillin plates, and the plasmid was checked for the presence of the p35 insert.

A SacI site present 5-bp from the 3′-end of the murine p35 cDNA sequence was used to ligate the nucleotide sequence encoding RGD-4C to the p35 cDNA. Two oligonucleotides encoding the forward and reverse DNA sequences were synthesized (Operon Technologies, Alameda, Calif.). The sequence for the sense oligonucleotide was 5′-CCG GGG AGC TCT GTG ACT GTC GAG GCG ACT GTT TTT GTT AAG ATA TCG G-3′ (SEQ ID NO:7); the sequence for the antisense oligonucleotide was complementary to SEQ ID NO:7. The sense and antisense oligonucleotides were annealed by mixing the oligonucleotides, heating to 80 C and rapidly cooling to allow annealing of the two strands. The homing peptide DNA was digested with SacI and EcoRV and cloned into the SacI site of p35 and the EcoRV site of the pSP72 vector harboring p35. The p35RGD-4C sequence was subcloned into the mammalian expression vector pcDNA3.1/Hygro+ (pcDNA3.1/p35RGD-4C).

The high p40-expressing CHO cell clones (C2 and C4) were transfected with pcDNA3.1/p35RGD-4C. Transfected cells were allowed to grow for 2 weeks in 60-mm tissue culture dishes in the presence of G418 (200 μg/mL) and hygromycin (300 μg/mL). Supernatants from the double-transfected CHO cell population were examined for mrIL-12vp by screening with a commercially available ELISA that is specific for the murine p70 IL-12 protein (OPTEIA™ ELISA kit, BD PHARMINGEN™). Supernatants from CHO cells expressing only the p40 molecule and supernatants from non-transfected CHO cells were used as negative controls to test for the presence of mrIL-12vp. Clones expressing the greatest concentrations of mrIL-12vp were expanded from colonies arising from single cells in selection medium containing 200 μg/mL of G418 and 300 μg/mL of hygromycin. Two clones, C2-A4 and C2-B3 were found to produce μg quantities of the fusion protein/10⁶ cells over a period of 24 hours.

### EXAMPLE 3

### Cloning and Expression of rIL-12vp from Other Species

The nucleic acid sequence encoding murine p35 subunit has a convenient SacI site located 5-bp from the 3′-end and methods for generating a fusion protein via this restriction site are exemplified supra. However, this site does not exist in all species. For example, neither human nor canine IL-12 contains this restriction site. Accordingly, alternative means for inserting the RGD sequence into an IL-12 subunit may be required for other species.

For example, to produce fusion proteins comprising human or canine IL-12, a 6-bp sequence encoding a KpnI restriction site can be incorporated, e.g., via PCR, into the sequence located between the end of the p35 gene and the beginning of a RGD-containing peptide (e.g., RGD-4C). This translates into an insertion of two amino acids, glycine and threonine, at the end of the p35 subunit. The sequence at the C-terminal end of the human and canine p35 subunits fused to RGD-4C as compared to the murine p35 subunit are as follows:
Human: MSYLNASGTCDCRGDCFC (SEQ ID NO:8)
Canine: MSYLNSSGTCDCRGDCFC (SEQ ID NO:9)
Murine: MGYLSSACDCRGDCFC (SEQ ID NO:10)
   wherein, the additional amino acids are in bold type and the RGD-4C homing peptide is underlined.

Thus, the ligation of RGD-4C to human IL-12 can be carried out as follows. RNA of the human p40 subunit is first extracted using TRIZOL® (INVITROGEN™) from peripheral blood lymphocytes (PBL) that have been stimulated for 40 hours with 0.0075% fixed SAC cells (Pansorbin, Calbiochem, La Jolla, Calif.). Reverse transcription can be carried out according to the manufacturer's instructions using the SUPERSCRIPT™ Preamplification System (Life Technologies, Inc.). The restriction enzyme sites for KpnI and EcoRV can be incorporated into the 5′- and 3′-ends of the PCR product, respectively, during the PCR amplification reaction. Accordingly, the primer sequence for the p40 sense strand is 5′-CCG GTA CCA TGT GTC ACC AGC AGT TG-3′ (SEQ ID NO:11) and the primer sequence for the antisense strand is 5′-CCG ATA TCC TAA CTG CAG GGC ACA GA-3′ (SEQ ID NO:12). The product can be amplified by 35 rounds of PCR using standard reagents and non-degenerate PCR conditions. The PCR product is then cut with the restriction enzymes KpnI and EcoRV and ligated into the vector pcDNA3.1/Neo(+) (INVITROGEN™; Carlsbad, Calif.), likewise digested with the same restriction enzymes. The rest of the procedure for isolating plasmids containing the p40 gene, purifying endotoxin-free plasmid for transfection, transfection of CHO cells, and isolation of CHO cell clones expressing high amounts of human IL-12 p40 subunit is carried out according to the methods described for the mouse clones. The two clones expressing the greatest amounts of p40 are then used in subsequent transfections with the p35 clone.

Isolation and expression of human IL-12 p35 cDNA from human cells is generally carried out as described above for the p40 subunit. However, the restriction enzyme sites for NheI and KpnI are incorporated into the 5′- and 3′-ends of the PCR product, respectively, during the PCR reactions. Thus, the primers for the p35 sense strand are 5′-CCG CTA GCA TGT GGC CCC CTG GGT CA-3′ (SEQ ID NO:13) and the primer sequence for the antisense strand is 5′-CCG GTA CCG GAA GCA TTC AGA TAG CT-3′ (SEQ ID NO:14). The product is amplified by 35 rounds of PCR using standard reagents and well-established non-degenerate PCR conditions. The amplicon is cut with the restriction enzymes NheI and KpnI and ligated into the vector pcDNA3.1/Hygro(+) (INVITROGEN™). Plasmid containing the p35 insert is generated as described supra.

For the ligation of the RGD-4C homing peptide to the human p35 subunit, two primers encoding the forward and reverse DNA sequences are synthesized (Operon Technologies, Alameda, Calif.). The sequence for the sense primer is identical to that used for the mouse system except different restriction sites are used, and the sequence for the antisense primer is complementary. Specifically, a KpnI site is incorporated into the 5′-end of the homing peptide DNA sequence and an XhoI site is incorporated into the 3′-end of the sequence. The primers are annealed as described supra. The plasmid containing the p35 sequence (p35 pcDNA3.1/Hygro (+)) is cut with the restriction enzymes KpnI and XhoI. The annealed oligonucleotides are also cut with these same enzymes. The products are then gel-purified and ligated. Transformation of JM109 cells, purification of endotoxin-free plasmid for transfection, transfection of CHO cells, and isolation of CHO cell clones expressing high amounts of human IL-12 is carried out according to methods described for the mouse clones. Concentrations of human recombinant IL-12 (hrIL-12) can be determined via ELISA.

A CHO expression system for canine IL-12 can be produced according to the methods disclosed herein for the human system with the following exceptions: the restriction site on the antisense primer is EcoRI, and the p40 subunit is ligated into the vector pcDNA3.1/myc-HisA. The change in restriction sites is necessary because the canine gene sequence contains an EcoRV restriction site and an EcoRV restriction digest would cleave the open reading frame. The canine p40 gene sequence does not contain an EcoRI site. The second change results in incorporation of a Histidine tag (His) onto the 5′-end of the p40 subunit of canine IL-12. This is necessary because there is currently no commercially available ELISA kit that can be used to detect the canine cytokine. However, an antibody for IL-12 is commercially available (R&D systems) for recognition of the canine protein (Helfand (1999) Cancer Res. 59:3119-3127). This antibody can be used as the capture antibody in an ELISA system, and an antibody that recognizes the His-tag on the p40 subunit can be used as the detection antibody.

The primers used to amplify the canine p40 and p35 sequences are as follows:
p40 sense: 5′-CCG GTA CCA TGC ATC CTC AGC AGT TG-3′ (SEQ ID NO:15); p40 antisense: 5′-CCG AAT TCA CTG CAG GAC ACA GAT GC-3′ (SEQ ID NO:16); p35 sense: 5′-CCG CTA GCA TGT GCC CGC CGC GCG GC-3′ (SEQ ID NO:17); and p35 antisense: 5′-CCG GTA CCG GAA GAA TTC AGA TAA CT-3′ (SEQ ID NO:18).

### EXAMPLE 4

### Protein Purification

Cells from the high mrIL-12vp expressing CHO cell clone, C2-A4, were cultured in DMEM/F12 (INVITROGEN™) supplemented with 5% (volume/volume) heat-inactivated fetal bovine serum, 2 mM L-glutamine, penicillin (100 units/mL) and streptomycin (100 μg/mL) at 37 C in a 5% CO₂ atmosphere until 80% confluent. The cells were then cultured in serum-free medium for 48 hours. Cell culture supernatants were harvested and enriched for mrIL-12vp using Vivacell 70 centrifugal filter devices (Sartorius AG, Goettingen, Germany) with a 50,000 molecular weight cut off. This partially enriched fraction of mrIL-12vp was used for initial experiments. To obtain the highly enriched protein used for later experiments, the concentrate was diluted 5× with PBS, pH 7.2 and applied to an antibody affinity column. The mrIL-12vp was eluted using 100 mM glycine, pH 3.0 and collected in tubes containing Tris buffer, pH 8.0. The fractions containing the highly enriched protein were combined and desalted. The protein was lyophilized and resuspended in PBS. For generation of the affinity column, antibodies recognizing the p40 subunit of IL-12 were harvested and purified. Antibodies were purified from cell culture supernatant using protein G affinity chromatography and eluted using a low pH buffer followed by desalting using a SEPHAROSE® bead column. Purity of mrIL-12vp was determined by SDS-PAGE followed by COOMASSIE blue staining. In addition, immunoblot analysis was performed to verify the presence of both the p35 and p40 subunits of IL-12. The amount of mrIL-12vp in the purified fractions was determined by ELISA (PHARMINGEN™). The molarity of mrIL-12 and mrIL-12vp used in the experiments was almost identical since the proteins differ in size by only a few amino acids. Thus, all calculations to determine the concentration of mrIL-12vp assumed that the protein had the same molecular weight as mrIL-12.

An RGD-4C peptide was synthesized and purified using standard Fmoc (N-(9-fluorenyl)methoxycarbonyl) chemistry according to standard methods (Clark, et al. (2001) J. Biol. Chem. 276:37431-37435), using a linear gradient of acetonitrile (0-80 minutes, 10-50%) at 3 mL/minute.

### EXAMPLE 5

### IFN-γ Assays

Assays to measure IFN-γ in vitro from mrIL-12- or mrIL-12vp-stimulated splenocytes from BALB/c mice were carried out in accordance with established methods (Schoenhaut, et al. (1992) J. Immunol. 148: 3433-3440), . . . and the concentration of IFN-γ was determined using an. IFN-γ OPTEIA™ ELISA kit (BD PHARMINGEN™). The induction of IFN-γ was also investigated in vivo in cohorts of 5-8 BALB/c mice treated with 1 μg/day of mrIL-12, mrIL-12vp, or with PBS by continuous infusion for 1, 3, 5, and 7 days using subcutaneously implanted osmotic pumps (ALZET®, model 2001, Durect Corporation, Cupertino, Calif.). Whole blood was collected by cardiac puncture from anesthetized mice on the days of interest, the sera separated, and stored at −20° C. Pumps were removed from each mouse at the time of blood collection, and the remaining volumes were measured to insure that there had been uniform delivery.

### EXAMPLE 6

### Toxicity Studies in Mice

DBA/2J mice were used in experiments to assess the toxicity of mrIL-12vp. Mice (five/group) received 0.025, 0.05, 0.1, 0.25, and 0.5 μg of mrIL-12 or mrIL-12vp per day by intraperitoneal (IP) injection or 0.1, 0.25 and 0.5 μg/day by continuous subcutaneous (SC) infusion using an osmotic pump. Mice were treated for two weeks and examined twice daily for signs of toxicity (weight loss, inappetence, ruffled fur, listlessness, etc). All mice were euthanized at the end of the treatment period or earlier if toxicity developed. Complete necropsy was performed on all mice, and microscopic examination of hematoxylin and eosin stained sections from formalin-fixed paraffin embedded tissues was performed by a board certified veterinary pathologist.

### EXAMPLE 7

### Immunofluorescence Labeling

Immunofluorescence labeling was performed in accordance with standard methods (Teng, et al. (2002) Am. J. Physiol. Renal Physiol. 282:F1075-F1083). M21 or Saos-2 cells were incubated with 5 μg of either mrIL-12 or mrIL-12vp for 30 minutes at 37 C in a 5% CO₂ atmosphere. The cells were rinsed with PBS, and then fixed with freshly prepared 4% paraformaldehyde for 10 minutes at room temperature. Detection of mrIL-12 or mrIL-12vp binding to cells used an anti-mouse IL-12 p40 antibody, followed by three washes with PBS and incubation with FITC-conjugated goat anti-mouse IgG (1:200) for 1 hour in the dark. As a control, anti-mouse IL-12 p40 was omitted from the labeling process.

### EXAMPLE 8

### Corneal Neovascularization Assay

Polyvinyl sponges pre-irradiated with 2000 cGy from a ¹⁵⁷Cs source were cut into 0.4×0.4×0.2 mm pieces, and 100 ng of bFGF, 200 ng of VEGF, or 2×10⁶ NXS2 murine neuroblastoma tumor cells were introduced into each sponge using a Hamilton syringe (Reno, Nev.). PBS was used as a negative control. The loaded sponges were air-dried, covered with a layer of 12% Hydron S, and then dried under a vacuum. Female adult BALB/c, DBA/2J, A/J, IFN-γ^{-/-}, or IL-12R^{-/-}mice were anesthetized with Avertin, and the sponges were introduced into a surgically created micropocket in an avascular area of one cornea. Two days later, mice were anesthetized and osmotic pumps containing either 200 μL of PBS, mrIL-12, or mrIL-12vp were implanted SC into cohorts of five to eight mice each. Pumps delivered 24 μL/day continuously for seven days, and doses of mrIL-12 and mrIL-12vp ranging from 0.25-1.0 μg/mouse/day were administered. After seven days of treatment, mice were anesthetized and 200 μL of FITC-conjugated high molecular weight dextran (3,000,000 MW, Sigma, St. Louis, Mo.) was injected into the tail vein, and the animals were euthanized three to five minutes later. Eyes were enucleated and fixed for 5 minutes with 4% paraformaldehyde. The cornea with the adjacent limbus was dissected from each eye, rinsed in PBS, and mounted with 10% glycerol onto a glass slide. Phase contrast and fluorescence microscopy (Stemi SV11, Zeiss, Thornwood, N.Y.) were used to visualize the overall appearance of the corneas and the presence of the perfused blood vessels (appearing green), respectively. Images were digitally recorded and, the corneal surface area (counted as the number of fluorescent green pixels) occupied by the vessels was calculated as a fraction of the total corneal area using Adobe Photoshop.

### EXAMPLE 9

### Murine Tumor Model

SC tumors were induced by injection of 2×10⁶ NXS2 murine neuroblastoma tumor cells in 200 μL of PBS in the right lateral flank. Once tumors were first palpable (11 days), mice were anesthetized and osmotic pumps containing either 200 μL of PBS, mrIL-12, or mrIL-12vp were implanted SC into cohorts of six mice each. Mice were monitored daily, and tumor growth was monitored weekly measuring SC tumors with calipers and determining the tumor volume using the formula, tumor size=(length)×(width)²×(n/6)=mm³. Mice were sacrificed 21-28 days later, or when they became moribund.

### EXAMPLE 10

### Statistics

All measurements were performed in duplicate and all experiments were repeated at least twice. Differences between experimental groups were evaluated with a Kruskal-Wallis test and, when significant, subsequent pair wise Wilcoxon tests. A P value less than 0.05 was considered statistically significant.

1. A fusion protein comprising a mammalian interleukin-12 operably linked to an RGD-containing peptide.

2. The fusion protein of claim 1, wherein the RGD-containing peptide comprises SEQ ID NO:2.

3. The fusion protein of claim 1, wherein the RGD-containing peptide consists of RGD.

4. A nucleic acid sequence encoding the fusion protein of claim 1.

5. A vector comprising the nucleic acid sequence of claim 4.

6. A host cell expressing the vector of claim 5.

7. A method for inhibiting growth of an angiogenic endothelial cell or an αᵥβ₃-positive tumor cell comprising delivering a mammalian interleukin-12 protein to an angiogenic endothelial cell or an αᵥβ₃-positive tumor cell via a fusion protein of claim 1 thereby inhibiting the growth of the angiogenic endothelial cell or the αᵥβ₃-positive tumor.

8. A method for decreasing toxic side effects associated with interleukin-12 administration in a mammal comprising generating a fusion protein comprising interleukin-12 and an RGD-containing peptide, and administering to a mammal the fusion protein thereby decreasing the toxic side effects associated with interleukin-12.

9. A method for treating cancer in a mammal comprising administering to a mammal having a cancer an effective amount of a fusion protein of claim 1 so that the cancer in the mammal is treated.


SEQUENCE LISTING (18 sequences)

SEQ ID NO: 1 - protein, 11 aa, Artificial Sequence
  RGD Peptide
ACDCRGDCFCG

SEQ ID NO: 2 - protein, 9 aa, Artificial Sequence
  RGD Peptide
CDCRGDCFC

SEQ ID NO: 3 - DNA, 26 bp, Artificial Sequence
  Synthetic Oligonucleotide Primer
ccggtaccatgtgtcttcagaagcta

SEQ ID NO: 4 - DNA, 26 bp, Artificial Sequence
  Synthetic Oligonucleotide Primer
ccgatatcctaggatcggaccctgca

SEQ ID NO: 5 - DNA, 27 bp, Artificial Sequence
  Synthetic Oligonucleotide Primer
ccggtaccatgtgtcaatcacgtctac

SEQ ID NO: 6 - DNA, 26 bp, Artificial Sequence
  Synthetic Oligonucleotide Primer
ccgatatctcaggcggagctcagata

SEQ ID NO: 7 - DNA, 49 bp, Artificial Sequence
  Synthetic Oligonucleotide
ccggggagctctgtgactgtcgaggcgactgtttttgttaagatatcgg

SEQ ID NO: 8 - protein, 18 aa, Artificial Sequence
  Synthetic Fusion Peptide
MSYLNASGTCDCRGDCFC

SEQ ID NO: 9 - protein, 18 aa, Artificial Sequence
  Synthetic Fusion Peptide
MSYLNSSGTCDCRGDCFC

SEQ ID NO: 10 - protein, 16 aa, Artificial Sequence
  Synthetic Fusion Peptide
MGYLSSACDCRGDCFC

SEQ ID NO: 11 - DNA, 26 bp, Artificial Sequence
  Synthetic Oligonucleotide Primer
ccggtaccatgtgtcaccagcagttg

SEQ ID NO: 12 - DNA, 26 bp, Artificial Sequence
  Synthetic Oligonucleotide Primer
ccgatatcctaactgcagggcacaga

SEQ ID NO: 13 - DNA, 26 bp, Artificial Sequence
  Synthetic Oligonucleotide Primer
ccgctagcatgtggccccctgggtca

SEQ ID NO: 14 - DNA, 26 bp, Artificial Sequence
  Synthetic Oligonucleotide Primer
ccggtaccggaagcattcagatagct

SEQ ID NO: 15 - DNA, 26 bp, Artificial Sequence
  Synthetic Oligonucleotide Primer
ccggtaccatgcatcctcagcagttg

SEQ ID NO: 16 - DNA, 26 bp, Artificial Sequence
  Synthetic Oligonucleotide Primer
ccgaattcactgcaggacacagatgc

SEQ ID NO: 17 - DNA, 26 bp, Artificial Sequence
  Synthetic Oligonucleotide Primer
ccgctagcatgtgcccgccgcgcggc

SEQ ID NO: 18 - DNA, 26 bp, Artificial Sequence
  Synthetic Oligonucleotide Primer
ccggtaccggaagaattcagataact